(19) United States
(12) Patent Application Publication
(10) Pub. No.: US 2024/0094232 A1
(43) Pub. Date: Mar. 21, 2024

(54) METHOD AND DEVICE FOR HANDLING BIOLOGICAL CELL CULTURES

(71) Applicant: AIXINNO Ltd., Cambridge (GB)

(72) Inventors: Patrick Clemente GILLIGAN, Cambridge (GB); Kenneth B. K. TEO, Cambridge (GB); John Edward CONNAH, Cambridge (GB); David Anthony BULLINARIA, Bluntisham (GB)

(21) Appl. No.: 18/275,158

(22) PCT Filed: Feb. 3, 2022
(86) PCT No.: PCT/EP2022/052551

§ 371 Date: Jul. 31, 2023

(30) Foreign Application Priority Data
Feb. 3, 2021 (DE) 10 2021 102 487.0

(51) Int. Cl. G01N 35/00; C12M 3/00

(52) U.S. Cl. CPC G01N 35/0099; C12M 23/48; G01N 35/00732; G01N 2035/00752; G01N 2035/00801; G01N 2035/00851

(57) ABSTRACT

A method and device for handling supports which support biological cell cultures or auxiliary agents for treatment of biological cell cultures, using one or more modules, at least one of which is a handling device. Each one of a plurality of supports rests with its edges on support elements, and, depending on the type of supports or the type of auxiliary means supported thereby, the supports require different storage heights, some of which are greater than one or more times a standardized vertical spacing of the support elements. The supports have an information carrier, the information of which can be read by a reading device and can be evaluated by a computing device. For optimal use of the compartments of the storage device, the information contains details about the storage height of the support in question and the handling of the supports is performed using these details.

### RELATED APPLICATIONS

This application is a National Stage under 35 USC 371 of and claims priority to International Application No. PCT/EP2022/052551, filed 3 Feb. 2022, which claims the priority benefit of DE Application No. 10 2021 102 487.0, filed 3 Feb. 2021.

### FIELD OF THE INVENTION

The invention relates to a method for handling supports, which carry biological cell cultures or auxiliary agents for treating biological cell cultures, with a handling device having an end effector, for example a gripper, wherein a plurality of supports each rests with its edges on support elements, which are allocated to each other in pairs and protrude at a standardized vertical distance from the opposing side walls of a storage device, and the supports, depending on their type or the auxiliary agents they carry, require different storage heights, some of which are greater than one or more times the standardized vertical distance, wherein the supports have an information carrier, the information of which can be read by a reading device allocated to the handling device, and can be evaluated by a computing device.

### BACKGROUND

WO 2020/098958 A1 describes a system for storing and handling biological cell cultures. The biological cell cultures are stocked on microplates. The microplates are in turn stocked in storage modules. The cell cultures are treated with auxiliary agents, for example with liquids, pipette tips and other objects, which are stocked on supports. The microplates form carriers for the biological cell cultures. The supports for the auxiliary agents have the same layout (surface area) as the supports for the cell cultures. In order to identify the supports, information carriers are provided, which can have a barcode, text, or RFID's. A reading device is provided, with which the information can be read.

The auxiliary agents can occupy different storage heights. The storage height essentially depends on the type of auxiliary agent. Supports that carry different auxiliary agents can be arranged in a uniformly designed module with several, in particular shelf-type storage devices. Each storage device has two facing vertical side walls, the distance between which is slightly greater than a width or length dimension of the support. Support elements protrude from the side walls, upon which the edge of the support can be placed. Pairs of respective support elements arranged one above the other at a standardized distance are provided, upon which the respective edge of a support, a frame of the support or a base section of the support can be placed. The supports can occupy storage heights that differ from each other. The storage height essentially depends on the type of auxiliary agent carried by the support. For example, bottles can occupy a higher storage height than platters. Pipette tip holders can in turn occupy a higher storage height than microplates. Supports with a high storage height occupy a plurality of compartments each defined by the distance between two support elements arranged one above the other. A microplate can occupy a single compartment.

U.S. Pat. No. 8,267,310 B2 describes a method for handling a storage unit for medicine.

### SUMMARY OF THE INVENTION

The object of the invention is to indicate measures that make it possible to optimally use the compartments of the storage device.

The object is achieved by the invention indicated in the claims, wherein the subclaims describe both advantageous further developments of the invention indicated in the ancillary claims, as well as independent solutions to the object.

It is initially and essentially proposed that the information carrier receive an indication about the storage height that can be directly ascertained with the reading device. The indication about the storage height can be a plain text indication which can be read via OCR recognition. However, the indication can also be coded in a barcode. It can also be the information content of an RFID. It is essential that the indication can be determined on its own, without interspersing external tables. The storage height can be the height of the support. However, it can also be the overall height of the support including the auxiliary agent carried by the support. In particular, it is provided that the handling device be controlled by a local control or computing device. This local control or computing device is preferably capable of reading a storage height directly from the information of the information carrier. The storage height is thus determined autonomously, without using external or internal tables or otherwise stored data, and instead only by reading the information carried by the information carrier. The method according to the invention initially comprises allocating information carriers to a respective support. The information carrier can carry various information, for example a date, details about the type of support, details about the type of auxiliary agent carried by the support, or details about the kind of biological cell culture carried by the support. In addition, the information carrier can carry a unique identification value or indicate an orientation. Furthermore, a test sum can be stored on the information carrier, and used to check the information. A support can carry several information carriers, for example two different, in particular opposing, sides can each carry an information carrier, wherein information carriers like these additionally carry information about the respective side, for example the “front side” or “back side”. However, it is essential that the information carrier contain an indication about the storage height. The information height can be understood as a height indication that denotes the entire height of the support, including the auxiliary agent it carries, for example in centimeters. However, the information height can also be an indication about the number of compartments of the storage device that are used for storing the support with the auxiliary agent it carries. The information carrier can be imprinted with the information. A sticker attached to the support can also be involved. Each support can have an individualized information carrier. The information carrier can also be a materially uniform part of the support, for example the information carrier can be on the surface area of the support provided with information. For example, the information can be embossed or engraved on a surface area of the support itself. In addition to information about the storage height, the information carrier can also contain information about what the support is carrying. The support can have a base section or a frame. The frame or base section can form support shoulders, which support the support on the support elements. The support elements can be support bars, which protrude slightly from the two opposing side walls. However, the support bars protrude into the free space between the side walls with enough width to allow the support to rest securely on the support bars. A horizontal free surface extends between two vertical supports running at the same height, which each are allocated to one of two facing side walls of a storage device, and has a large enough cross section to allow what the support is carrying to protrude through there. There is an exemplary distance of 30 mm between support bars arranged one vertically above the other, which represents a standardized distance dimension that defines the height dimension of a compartment. The horizontal distance between two support bars lying in a plane is here dimensioned in such a way that the auxiliary agents can extend over several compartment heights. The information carrier can be arranged at a defined location, for example a specific surface section of a base section or a frame of the support can carry the information carrier. One or several modules are preferably provided, wherein the modules are fixtures in which at least two supports can be accommodated. A handling device or a gripper or a handling device with a gripper can be allocated to at least one of the modules. Each module can have one or several storage devices. The storage device preferably has a removal side. The support can be removed from the storage device on this removal side. This preferably takes place with an end effector of the handling device. The end effector can consist of a shovel-like plate or a paddle, which can be moved under the support so as to reach under and lift the support. However, it is also possible that the end effector be a gripper with fingers that can be moved toward each other like pincers. The fingers can be used to grip the support. The storage device can have a removal side that faces an end effector. The storage devices can be arranged around a free space of a storage module. The storage devices can be shelflike in design, with several compartments arranged one above the other. Several identical storage devices can be arranged around the free space. The respective removal side faces toward the free space. The handling device can be arranged inside of the free space, and shifted in a vertical direction. The handling device can have a gripper that can be shifted in a horizontal direction, so that a support can be removed from the storage device or a support can be inserted into the storage device. According to a preferred embodiment of the invention, the gripper has a reading device, with which the information of the information carrier can be read. For example, the reading device can be a barcode scanner. However, the reading device can also be an imaging system, for example a camera, for example for reading plain text information. However, the reading device can also be an antenna for reading an RFID. Another embodiment can provide that the gripper transport the support to a reading device, which is arranged at some other location, and which can read the information carrier. It can be provided that the reading device be separate from the gripper, wherein a gripper is understood as any device with which the support can be moved.

A further development of the invention provides that a proximity sensor be provided. The proximity sensor can be allocated to the gripper. It can be arranged adjacent to the reading device. The proximity sensor can be used to detect whether a compartment is occupied by a support, for example by a frame of a support or a base section of the support. However, the proximity sensor can also be used to detect whether a compartment arranged above a base section is occupied by an auxiliary agent or the like. The proximity sensor can be an optical sensor, an ultrasound sensor, or also a capacitive or inductive proximity sensor. The proximity sensor can also be a laser proximity sensor, a camera, in particular an additional camera. However, it is also provided that the reading device be designed in such a way that it can perform the function of a proximity sensor. The proximity sensor and the reading device can be arranged on the gripper.

The method according to the invention makes it possible to optimize the use of the compartments of a storage device. A local control or computing device of a module that is equipped with several storage devices of the kind described above and has a gripper can be used to control the gripper in such a way that it removes individual supports from the storage device and reinserts them into the same storage device or another storage device at another location. The gripper can use its reading device and proximity sensor to determine free compartments beforehand. By continuing to remove individual supports and reinsert the individual supports at another location of the module, the supports having different storage heights can be arranged in such a way as to increase the total number of free compartments arranged one directly above the other. For example, a single free compartment can be filled by using the gripper to search for a support that has a storage height of a height unit. This support can fill the individual free compartment. Alternatively thereto, however, a support that has a storage height of several height units and is arranged directly below the free compartment or directly above the free compartment can be raised or lowered, so as to thereby fill the free compartment. In an analogous manner, several free compartments arranged one above the other can be filled by using the proximity sensor to initially determine the height of the free space formed by the free compartments. For this purpose, the gripper approaches the positions at which the information carrier can usually be found. If no information carrier can be found, and the proximity sensor does not give a signal, it can be assumed that the compartment is not occupied. In this way, a plurality of free compartments arranged one above the other can be determined. The reading device can then be used to search for a support in the storage devices that has a storage height corresponding to the height of the free compartments arranged one above the other. According to the invention, this takes place by reading the information carrier. According to the invention, the latter has the indication about the storage height. The found support can then be inserted into the free compartments by means of the gripper.

The invention can also provide that the storage devices of a module be designed identically to each other. For example, the storage devices can be arranged in a kind of carousel, and turned by a rotary drive. A side wall of the module can form a uniform housing, and have a loading opening with which the module adjoins a process module or another storage module. Supports can be moved from one module into an adjacent module through the loading opening. The loading opening can be closable in a suitable manner. Each of two adjoining modules can have loading openings. The loading openings of two adjoining modules align with each other, so that supports can be moved through the two opened loading openings from one module to another module. In particular, the loading opening can be closable in such a way as to decrease a gas exchange between an interior side of the module or an exterior side of the module. The loading opening can also be closable in such a way as to decrease a heat flow between the exterior of the module and interior of the module. For example, the module can be an incubator or a freezer or a refrigerator. A preferred embodiment provides that a number of four, five, six, seven, eight, nine, ten, eleven or twelve storage devices be arranged around a central free space of the module in such a way that each storage device directly adjoins an adjacent storage device, so that a transfer of a support from one module to an adjacent module must take place via a storage device. In order to transport a support from inside of the module through the loading opening and out of the module, an area of a storage device lying in front the loading opening, for example a storage shelf, must form a free space through which the support is transported by means of the gripper. This free space must have at least the vertical height that corresponds to the storage height of the support. The gripper designed according to the invention can use the reading device and/or proximity sensor to determine whether a free space with a sufficient vertical height is available. In a case where such a free space is not available, it is proposed that the gripper keep removing supports from an area of a storage device lying in front of the loading opening until a free space with sufficient vertical height becomes available, wherein the vertical height corresponds to at least the height of the support to be transported through the loading opening. It can be provided that all storage devices of a module are basically suitable for providing a free space, and the control device is set up in such a way in order to form the free space as to select the storage device that requires a minimum number of supports to be moved to form a large enough free space, through which a selected support with the detected storage height can be moved.

### BRIEF DESCRIPTION OF THE DRAWINGS

Exemplary embodiments of the invention will be described below based on the attached drawings. Shown on:

FIG. 1 is an exemplary perspective view of eight different supports for using biological cell cultures,

FIG. 2 is a schematic view of a device for treating biological cell cultures,

FIG. 3 is the section through a storage module as depicted on FIG. 2 with reference numbers 12, 13 and 14,

FIG. 4 is a schematic view of a storage shelf 19 and a handling device 22 with a gripper (end effector) 23, which has a reading device 24 and a proximity sensor 25,

FIG. 5 is a schematic view of an area of two modules 12, 14 in the area of a loading opening 26 that connects the two modules 12, 14 with each other,

FIG. 6 is a magnified cutout of two storage devices 19 arranged side by side.

### DETAILED DESCRIPTION

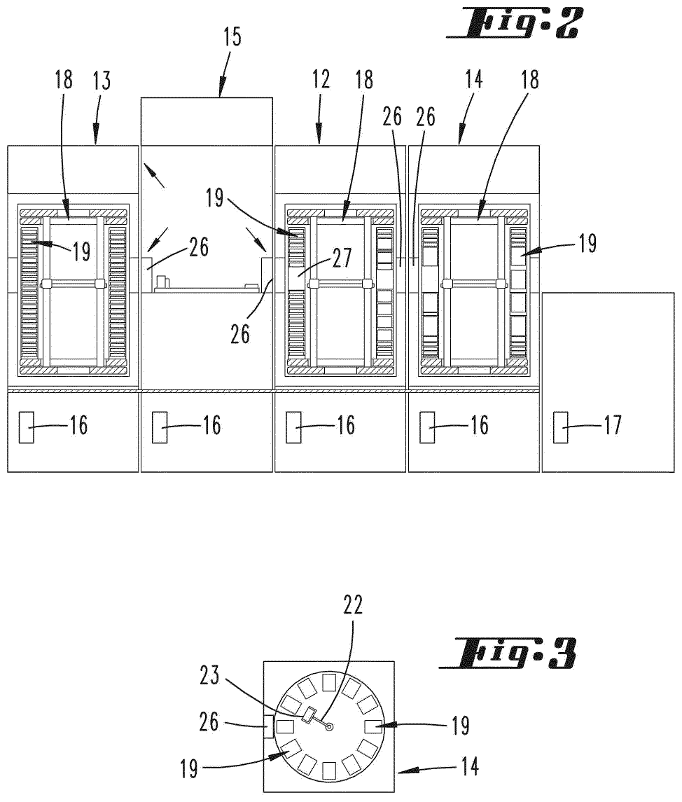

FIG. 2 presents a schematic view of three storage modules 12, 13, 14 for receiving standardized supports, which can accommodate cell cultures or auxiliary agents for their treatment. A storage module 12 is used to receive auxiliary agents for treating cell cultures. However, microplates that carry cell cultures can basically also be received in this storage module 12. A process module 15 is located between the storage module 12 and the storage module 13, which is an incubator, for receiving cell cultures. The cell cultures in this process module 15 can be treated by means of pipettes or the like. The storage module 14 is a refrigerator, in which treatment liquids can be stored that can be used to treat the cell cultures. The modules 12-14 as well as the process module 15 each have a local control device, which consists of a control or computing device 16. The local control devices 16 can have a data exchange link with a central control device 17.

The storage modules 12, 13, 14 are identical in design to each other, and essentially differ only in terms of the climate generated therein. A high temperature can prevail in the refrigerator 14. The storage modules 12, 13, 14 are interconnected via loading openings 26. The storage modules each have one or two loading openings 26. The loading opening 26 of a storage module can directly adjoin the loading opening of an adjacent storage module 14, so that supports 1-9 are transported from one storage module 12 into the other, adjacent storage module 14 with the loading openings 26 opened. In particular, it is provided that the loading openings 26 of directly adjacent storage modules be directly connected with each other. It is also provided that the loading openings 26 of a storage module 12 directly adjoin a process module. The loading openings 26 can be closed.

FIG. 3 shows the cross section through one of the storage modules 12, 13, 14. Several storage devices 19 are arranged around a center like a carousel. The storage devices 19 can be turned around the center, so that the storage device 19 can be positioned so as to align with a loading opening 26.

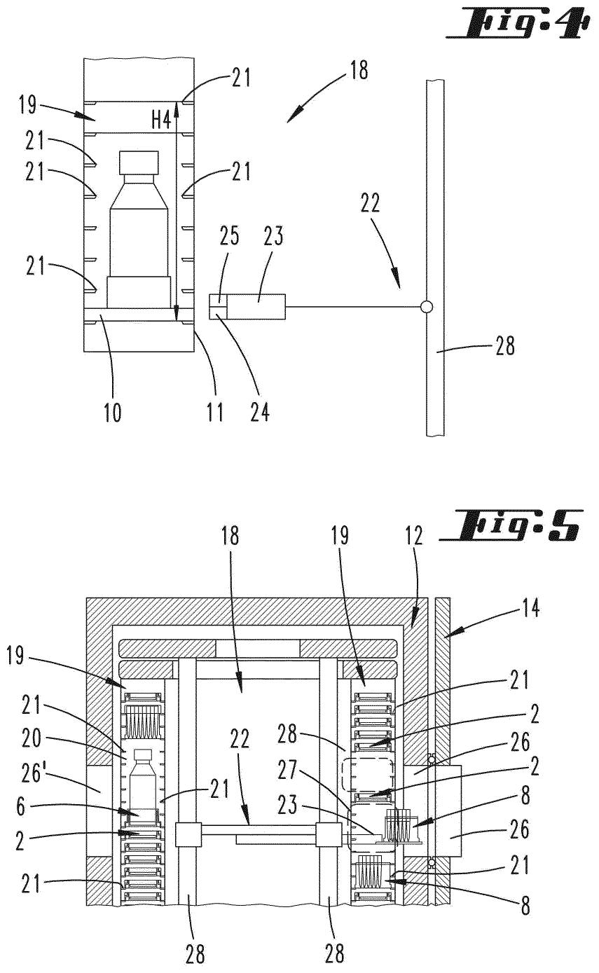

FIG. 4 presents a rough, schematic view of the structure of a storage device 19. The storage device 19 forms a storage shelf, in which supports 1-9 can be placed in several levels arranged one above the other. The storage device 19 has two side walls that run parallel to each other. Support elements 21 are secured to the facing side walls, in particular in the form of support bars. The horizontal distance between the support bars 21 can measure 5 cm to 15 cm, or also 20 cm to 35 cm. The horizontal distance between the support bars 21 is preferably adjusted to the length or the width of a standard microplate (85.48 mm×127.76 mm). The vertical distance between the support bars 21 lying opposite each other in pairs can be slightly greater than the height of the support 1, 2 having the lowest vertical height.

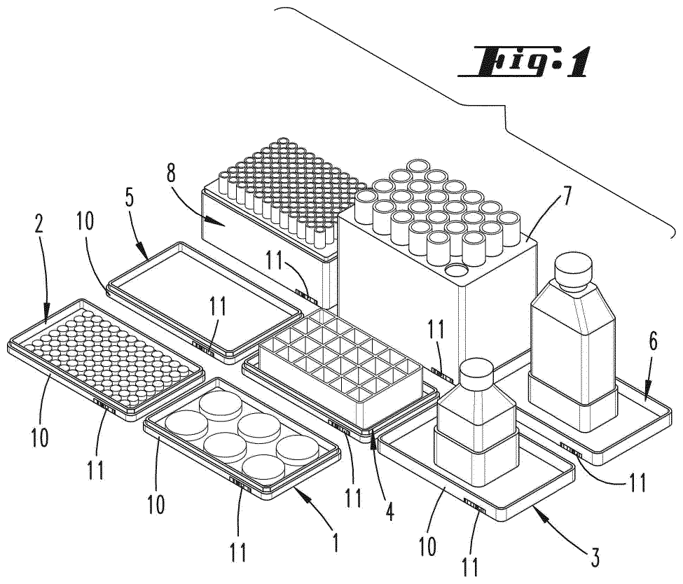

FIG. 1 exemplarily shows various supports 1-9, which are used for receiving cell cultures or auxiliary agents for their treatment. The supports 1, 2 are flat, rectangular bodies with recesses, in which biological cell cultures can be arranged. These types of supports can have a height of 21 mm. The support 1 has a base section 10, which can be a base body with a flat shape, the height of which is smaller than the distance between two support bars 21. This support 3 carries a bottle with a treatment liquid, for example which can be held in the refrigerator 14.

The support 4 carries a plurality of containers for receiving liquids or equipment or the like. The vertical height of the support 4 is greater than the vertical distance between two support bars 21 arranged one directly above the other.

The support 5 is a flat shell, and has a height of less than the vertical distance between two directly adjacent support bars 21. In particular, the support 5 is an individual container in which biological cell cultures can grow, or in which a liquid can be stocked.

The support 6 is similar to the support 3. It carries a bottle that can hold a larger volume than that carried by the support 3.

The support 7 carries a plurality of pipette tips or oblong containers for receiving liquids. The height of the support 7 is greater than the vertical distance between two directly adjacent support bars.

The support 8 carries a plurality of pipette tips or other oblong containers for receiving liquids. The height of the support 8 is greater than the vertical distance between two directly adjacent support bars 21.

FIG. 6 additionally shows a support 9 which is designed similarly to the support 5, but has an edge that surrounds a central recess and has a height greater than the vertical distance between two adjacent support bars. It can have a height of 30 mm to 40 mm.

The above statements serve to explain the inventions covered by the application as a whole, which each also independently advance the prior art at least by the following feature combinations, wherein two, several or all of these feature combinations can also be combined, specifically:

All supports 1-9 have a lower section 10, which is a base section, and carries an information carrier 11 at a standardized location. The information carrier 11 is arranged on the outer wall of the support 1-9. The information carrier 11 is arranged so as to be visible from a central cavity, which is surrounded by several storage devices 19. It can be provided that each support 1-9 has a single information carrier 11. However, it can also be provided that individual supports 1-9 each have several information carriers 11.

Additional exemplary embodiments of the invention can provide that sides of the support 1-9 facing away from each other can each have an information carrier 11. Other exemplary embodiments of the invention can provide that each side of the support 1-9 has an information carrier 11. In exemplary embodiments where the supports 1-9 have several information carriers 11 on different sides, it can be provided that each information carrier 11 also has information about the respective side of the support 1-9, i.e., information about whether a short side, a long side and/or a front or rear side of the support is involved, for example. It is preferably only required that it be possible to distinguish between the front and rear side. The information carrier can be a surface section of a plastic surface of a plastic body comprising the support.

FIG. 6 shows several supports 2, 5, 6, 8, 9 with different heights. Only the supports 2, 5 have a storage height H₁ that is less than the vertical distance between two support bars 21.

The distance between two support bars 21 lying one directly above the other defines a standard distance A. The support 9 has a storage height H₂ that is less than two times a standard distance A. The support 8 has a storage height H₄ that is greater than three times a standard distance, but less than four times a standard distance A, so that the support 8 has a storage height H₄ that takes up four compartments 20, wherein each compartment 20 is defined by the space surrounded by two support bars 21 lying one above the other. The support 6 carrying a bottle has a storage height H₇, and takes up seven compartments 20 lying one above the other.

The gripper 23 schematically shown on FIG. 4 is capable of gripping a support 1-9, for example with a plate that reaches under a support 1-9 or with gripping arms, so as to pull it out of the storage device 19 and either move it into another position in the storage device 19 or to move the support 1-9 through the loading opening 26 into an adjacent storage device 19 of another module 12-14.

The gripper 23 or the handling device 22 can have a reading device 24. This reading device 24 can be used to read the information of the information carrier 11. According to a preferred exemplary embodiment, the information is applied to the information carrier 11 as a barcode. The reading device 24 can be a barcode reader, which can be used to read the information of the barcode. The barcode can be arranged at a distance of within 10 mm, 20 mm, or 30 mm from the lower edge of the base section 10 of the support 1-9. It is readable if the support 1-9 is located in the storage devices. The base section 10 here rests directly on the support bars 21. Since all supports 1-9 have the same layout, specifically preferably that of a microplate, supports 1-9 with a varying storage height can be arranged in all storage devices 19.

In exemplary embodiments that are not shown, the reading device 24 can be arranged at another location inside of the storage module 14. In order to be able to read the information carrier 11 with this type of reading device 24 not arranged on the gripper 23 or the handling device 22, the support 1-9 carrying the information carrier 11 is transported to the reading device 24.

The information of the information carrier 11 contains a direct indication about the storage height H₁ to H₇. An absolute height indication provided in centimeters can be involved. However, a height indication that merely denotes a multiple of a standard distance A or height units can also be involved. It is essential that the height indication can be derived directly from the information carrier 11 without the assistance of additional tables or the like. As a consequence thereof, a local control or computing device 16 allocated to each of the modules 12-14 can receive an indication about the height of a support 1-9 from the information obtained from the reading device 24. The information carried by the information carrier 11 can additionally be a date, the type of auxiliary agent (labware). The additional information can further be a serial number, a uniform identifier, an orientation, or a checksum. For example, the computing device 16 can determine a bottle size or the size of a pipette tip from the type designation.

The barcode of the information carrier 11 can have the format LLLLLLL CCCCCCC UUUUUU O H C. Here, LLLLLLL is a batch number, CCCCCCC is a catalog number UUUUUU is a serial number, O is an orientation, H is the height or number of claimed standard distances, and C is a checksum. Other configurations are also possible. Alternatively, the barcode can have the format DDDDDDFFFFUUUUUUOHHCC, wherein DDDDDD is a current date, for example a production date. The current date can have the format YYYYWW, wherein YYYY is the year and WW is the calendar week, for example of production. FFFF can be the type of auxiliary agent. UUUUUU can be the serial number. O can be the orientation. HH can be the height, and CC can be a checksum.

The gripper 23 of the handling device 22 can carry not just a reading device 24, but also a proximity sensor 25. While the reading device can be used to read the information of the information carrier 11, i.e., in particular the bar code of the barcode, the proximity sensor 25 can be used to determine whether a compartment between two support bars 21 arranged one above the other is occupied.

The reading device 14 in combination with the proximity sensor 25 can be used to perform a control function. If the proximity sensor 25 determines a base section 10 arranged in a compartment, and if the reading device 24 there determines a barcode, the local control device 16 recognizes that the lower section of a support is here involved. The control device 16 recognizes how many compartments above the base section 10 are occupied by the same support from the height indication contained in the information of the information carrier 11. The proximity sensor 25 can be used to check whether this is the case, and whether the information of the information carrier is correct with regard to the storage height H₁ to H₇. In addition, a reading device 24 combined with a proximity sensor 25 can be used to determine whether a compartment is free. It is also possible to recognize a defective information carrier 11 if a compartment which is in itself free above the storage height of the support is not free and carries no barcode.

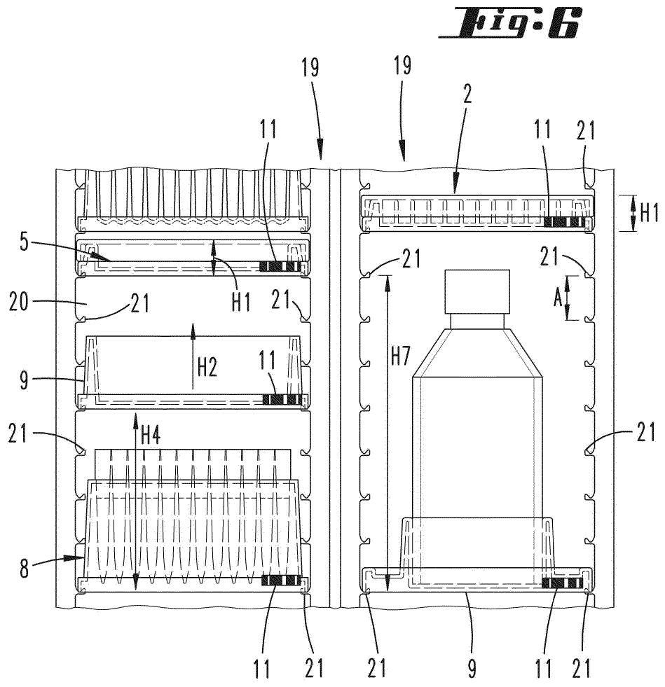

By labeling the supports 1-9 according to the invention with an information carrier 11 that is designed in particular as a barcode and has an explicit indication about a storage height H₁ to H₇, each storage module 12-14 can operate autonomously. No external data link is required to determine which storage height a support 1-9 has. As a consequence, it can be determined whether a transfer opening 27 in one of the storage devices 19 is available for transferring a support 1-9 through the loading holes 26′, 26 between two adjacent modules 12-14, through which the supports 1-9 can be transported. For example, the right side of FIG. 5 shows a loading opening 26, which has a larger vertical extension than the transfer opening 27 between a support 2 and a support 8 that is located behind it. The vertical height of the transfer opening 27 is large enough to transport a pipette tip box 8 through. However, the height of the transfer opening 27 is not sufficient for transporting a support 9 with a high bottle, for example. In order to enable the latter, it is necessary beforehand that the effective height of the transfer opening 27 be increased by removing the support 2 from the area of the loading opening 26. For this purpose, the support 2 is gripped by the gripper 23, and moved into another suitable position inside of the module.

In the situation shown on the left of FIG. 5, flat supports 2 arranged under the support 9 must initially be removed, so that the support 9 with the bottle can be transported through the loading opening 26′.

Therefore, the invention relates to a method, characterized in that the vertical height of a transfer opening 27 is determined with the help of the reading device 24 and/or even without the proximity sensor 25, and the vertical height of the transfer opening 27 is increased as necessary by removing supports 1-9 arranged there and arranging the supports 1-9 in other storage devices 19 or at other positions of the same storage device 19. This method initially involves identifying one of the supports 1-9 that is arranged in the storage module 12 and is to be removed from the storage module. The storage height H₁ to H₇ of the support 1-9 is then determined with the reading devices 24. The storage height H₁ to H₇ determined in this way is used to calculate the required vertical height of the transfer opening 27. The reading device 24 and possibly the proximity sensor 25 are then used to determine the actual vertical height of the transfer opening 27. If the actual vertical height of the transfer opening 27 is less than the storage height H₁ to H₇ of the selected support 1-9, the vertical height of the transfer opening 27 is increased by removing supports 1-9 arranged in the area of the loading opening 26.

By labeling the supports 1-9 according to the invention using an information carrier that is designed in particular as a barcode and has an explicit indication of a storage height H₁ to H₇, a storage module 12-14 can also organize itself automatically. This can take place even without the use of the proximity sensor 25. The reading device 24, which is preferably fastened to the gripper 23, but also to a separate support that can be moved independently of the gripper 23, can be used to determine the positions of the base sections 10 of all supports 1-9 arranged in a storage module 12. A storage module 12 can have a plurality of storage devices 19, in which supports 1-9 are located. The information about the storage height H₁ to H₇ can be used by the local computing device 16 to determine the actually occupied compartments 20, and in particular to calculate where free compartments 20 are located. By removing the supports 1-9 and arranging them at another location in the same storage module 12, the number of free individual compartments 20 can be decreased. By moving the supports 1-9 to other positions, the number of free spaces comprising several free compartments for arranging supports 1-9 with a large storage height H₁ to H₇ can be increased.

Therefore, the invention relates to a method, characterized in that the reading device 24 is used to decrease the number of free individual compartments 20 or to increase the number of directly adjacent free individual compartments 20, in which supports 1-9 are moved to other positions.

The invention further relates to a method and a correspondingly designed device, wherein the supports 1-9 arranged in a storage module can only be organized with the assistance of a reading device and a gripper, as well as the control and computing device 16. The reading device can be used to determine the lowermost compartment filled by a support. Determining the storage height makes it possible to then determine the next compartment lying above the support that is not claimed by a good carried by the support. The reading device can then be used to determine whether this compartment is free, and whether additional compartments lying above it are free. The gripper can subsequently be used to grip the support lying under free compartments, and upwardly displace it in such a way that no free compartments are present directly above the support. If such a method is initiated with the uppermost support, all free compartments are filled one after the other. Therefore, the method also relates to a method in which free compartments are sequentially determined between two supports lying one directly above the other starting either from the top or bottom, and in which these are filled by relocating the underlying or overlying support.

The invention further relates to a method in which the reading device 24 and the information about the storage height arranged on the information carrier 11 are used to find a storage space that has a prescribed minimum number of individual compartments 20 arranged one directly above the other.

In the above description, the term gripper 23 or handling device is construed as any type of means which, whether by itself or combined with other means, makes it possible to move a support from a first position into another, second position. As a consequence, the term gripper or handling device is also understood in particular as an end effector. In addition, the term gripper or handling device can also be understood as an electrically activatable device that can be controlled by a control device, with which a support 1-9 can be removed from a compartment 20 or placed inside of a compartment 20. However, a gripper 23 or handling device can also be understood as a device with which a support 1-9 can be removed from a compartment 20 by having the device or part of the device perform a horizontal movement. The device can also perform a vertical movement, so as to lift or lower the support 1-9. In addition, the gripper 23 or handling device can consist of a device with which a support 1-9 can be brought through two adjoining loading openings 26 from a storage module 12 into a storage module 14, or a support 1-9 can be transported between a storage module 12, 13 and a process module 15.

The invention also comprises those kinds of exemplary embodiments in which a plurality of storage modules 12-14 has at least one handling device 22, wherein the handling device 22 is capable of gripping or transporting at least one respective support 1-9. As a consequence, the invention also relates to a method with one or several modules 12-14, of which at least one has a handling device 22.

The above statements serve to explain the inventions covered by the application as a whole, which each also independently advance the prior art at least by the following feature combinations, wherein two, several or all of these feature combinations can also be combined, specifically:

A method for handling supports 1-9, which carry biological cell cultures or auxiliary agents for treating biological cell cultures, with a handling device 22 having a gripper 23, wherein a plurality of supports 1-9 each rests with its edges on support elements 21, which are allocated to each other in pairs and protrude at a standardized vertical distance A from the opposing side walls of a storage device in a horizontal direction, and the supports 1-9, depending on their type or the auxiliary agents they support, require different storage heights H₁-H₇, some of which are greater than one or more times the standardized vertical distance A, wherein the supports 1-9 have an information carrier 11, the information of which can be read by a reading device of the handling device 22, and can be evaluated by a computing device 16, characterized in that the information contains details about the storage height H₁-H₇ of the respective support 1-9, and these details are used for handling the supports 1-9.

A method, characterized in that the information carrier 11 is arranged on a base section 10 or frame of the carrier 1-9 having two support shoulders that can be placed on the support elements 21, such that the information can be read from a removal side of the storage device 19;

A method, characterized in that the handling device 22 has a proximity sensor 25, which is used to determine whether a compartment 20 extending vertically above a pair of support elements 21 and up to the pair of support elements 21 lying thereover is occupied, and/or whether a support 1-9 is resting on a pair of support elements 21;

A method, characterized in that the reading device 24 and the proximity sensor 25 are used to recognize compartments 20 occupied by supports 1-9 and free compartments 20, and to find a vertical arrangement of free compartments 20 corresponding to at least the storage height H₁-H₇ of the support 1-9 so as to add a support 1-9 in a storage device 19;

A method, characterized in that the vertical arrangement of free compartments 20 is found exclusively by using the reading device 24 and the proximity sensor 25;

A method, characterized in that the supports 1-9 arranged in a storage device 19 are removed from the compartments 20 to which they are allocated and allocated to other compartments 20 in such a way as to form new arrangements of compartments 20, in which the number of free compartments 20 arranged one directly above the other is increased;

A method, characterized in that a vertical area 27 of the storage device 19 is kept free at the height of the loading opening 26, the height of which corresponds to the largest storage height H₁-H₇ of a support 1-9 arranged in the housing 12-14;

A method, characterized in that the reading device 24 is allocated to the handling device 22, or is arranged in at least one of the modules 12-14, wherein the modules 12-14 are set up to stock a plurality of supports 1-9;

A device, characterized in that the information contains details about the storage height H₁-H₇ of the respective support 1-9, and an in particular programmable control device 16 is provided, which is set up in such a way that the handling of supports 1-9 takes place using these details;

A device, characterized in that a gripper 23 is allocated to the handling device 22, and/or that the reading device 24 is allocated to the handling device 22 or gripper 23;

A support, characterized in that the information of the information carrier 11 contains explicit details about the storage height H₁-H₇ of the respective support;

A support, characterized in that the information contains a date, details about the type of support 1-9, a unique identification value, an orientation and/or a checksum and/or a “batch number” and/or a value for a height;

A device, a method or support, characterized in that the information carrier 11 carries a barcode, and the reading device is a barcode scanner;

A device, a method or support, characterized in that a support 1-9 has several information carriers 11, and/or that at least two adjacent or opposing sides each have an information carrier 11, and/or that the information carriers 11 of supports 1-9 having several information carriers 11 additionally have information about the side to which the respective information carrier 11 is fastened.

All disclosed features (whether taken separately or in combination with each other) are essential to the invention. The disclosure of the application hereby also incorporates the disclosure content of the accompanying/attached priority documents (copy of the prior application) in its entirety, also for the purpose of including features of these documents in claims of the present application. Even without the features of a referenced claim, the subclaims characterize standalone inventive further developments of prior art with their features, in particular so as to submit partial applications based upon these claims. The invention indicated in each claim can additionally have one or several of the features indicated in the above description, in particular those provided with reference numbers and/or indicated on the reference list. The invention also relates to design forms in which individual features specified in the above description are not realized, in particular if they are recognizably superfluous with regard to the respective intended use, or can be replaced by other technically equivalent means.

Reference List

| | |
|---|---|
| 1 | Support, for flat shells |
| 2 | Support, microplate |
| 3 | Support, bottle carrier |
| 4 | Support, for containers |
| 5 | Support, with high tub |
| 6 | Support, bottle carrier |
| 7 | Support, for pipette tips |
| 8 | Support, for pipette tips |
| 9 | Support, with high tub |
| 10 | Base section |
| 11 | Information carrier |
| 12 | Storage module, for auxiliary agents |
| 13 | Storage module, for cell cultures, incubator |
| 14 | Storage module, for cell cultures, cooled |
| 15 | Process modules |
| 16 | Local control device, computing device |
| 17 | Central control device |
| 18 | Storage chamber |
| 19 | Storage shelf, storage device |
| 20 | Compartment |
| 21 | Support bar, element |
| 22 | Handling device |
| 23 | Gripper |
| 24 | Reading device |
| 25 | Proximity sensor |
| 26 | Loading opening |
| 26′ | Loading opening |
| 27 | Vertical area, transfer opening |
| 28 | Column |
| A | Standard distance |
| H₁ | Storage height |
| H₂ | Storage height |
| H₄ | Storage height |
| H₇ | Storage height |

1. A method for handling supports (1-9), which carry biological cell cultures or auxiliary agents for treating biological cell cultures, wherein a device for handling the supports (1-9) comprises one or more modules (12-14), of which at least one has a handling device (22), wherein the supports (1-9) each rests with its edges on support elements (21), which are allocated to each other in pairs and protrude at a standardized vertical distance (A) from the opposing side walls of a storage device (19) in a horizontal direction, and the supports (1-9) have respective storage heights (H₁-H₇) that are different from one another some of which are greater than one or more times the standardized vertical distance (A), and wherein the supports (1-9) each have an information carrier (11), the method comprising:
reading by a reading device 24 information from each of the information carriers (11);
evaluating by a computing device (16) the information from each of the information carriers (11), wherein the information contains details about the restive storage heights (H₁-H₇) of the supports (1-9); and
handling the supports (1-9) using the details contained in the information.

2. The method of claim 1, herein the information carrier (11) is arranged on a base section (10) or frame of each of the supports (1-9), each of the supports (1-9) having two support shoulders that are placed on the support elements (21), such that the information is read from a removal side of the storage device (19).

3. The method of claim 1, wherein the handling device (22) has a proximity sensor (25), the method further comprising using the proximity sensor (25) to determine at least one of:
(i) whether a compartment (20) extending vertically above a first pair of the support elements (21) and up to a second pair of support elements (21) lying over the first pair of support elements (21) is occupied; or
(ii) whether one of the supports (1-9) is resting on the first pair of the support elements (21).

4. The method of claim 3, further comprising using the reading device (24) and the proximity sensor (25) to recognize compartments (20) occupied by the supports (1-9) and free compartments (20), and to find a vertical arrangement of the free compartments (20) corresponding to at least a storage height (H₁-H₇) of a support (1-9) to be added in a storage device (19).

5. The method of claim 4, wherein the vertical arrangement of the free compartments (20) is found exclusively by using the reading device (24) and the proximity sensor (25).

6. The method of claim 1, further comprising removing the supports (1-9) arranged in the storage device (19) from compartments (20) to which they are allocated and allocating the supports (1-9) to other compartments (20) in such a way as to form new arrangements of the compartments (20), in which a number of free compartments (20) arranged one directly above another is increased.

7. The method of claim 1, wherein the storage device (19) is arranged in a housing (12-14) between a loading opening (26) of the housing (12-14) and the handling device (22), the method further comprising keeping a vertical area (27) of the storage device (19) free, a height of the vertical area (27) being equal or greater than a height of the loading opening (26) which corresponds to a largest one of the respective storage heights (H₁-H₇) of the supports (1-9) arranged in the housing (12-14).

8. The method of claim 1, wherein the reading device (24) is allocated to the handling device (22), or is arranged in at least one of the modules (12-14), and wherein the modules (12-14) are configured to contain the supports (1-9).

9. A device for handling supports (1-9), the device comprising:
a storage device (19) with support elements (21) that protrude in pairs at a standardized vertical distance (A) from respective side walls of the storage device (19);
a plurality of biological cell cultures or auxiliary agents for treating the supports (1-9) carrying biological cell cultures;
a handling device (22) for handling the supports (1-9), wherein each of the supports (1-9) has an information carrier (11);
a reading device (24) for reading information from each of the information carriers (11);
a computing device (16) for evaluating the information from each of the information carriers (11), wherein the information contains details about the restive storage heights (H₁-H₇) of the support (1-9); and
a programmable control device (16) for using these details to control the handling device (22).

10. The device of claim 9, further comprising a gripper (23) that is allocated to the handling device (22), wherein the reading device (24) is allocated to either the handling device (22) or the gripper (23).

11-12. (canceled)

13. The device of claim 9, wherein each of the information carriers (11) comprises a barcode, and the reading device (24) is a barcode scanner.

14. The device of claim 9, wherein at least one of:
(i) one of the supports (1-9) has several one of the information carriers (11);
(ii) two adjacent or opposing sides of one of the supports (1-9) each has one of the information carriers (11); or
(iii) the information carriers (11) of the supports (1-9) having several information carriers (11) additionally comprise information regarding a side to which the respective information carrier (11) is fastened.

15. (canceled)

16. The method of claim 1, wherein each of the information carriers (11) comprises a barcode, and the reading device is a barcode scanner.

17. The method of claim 1, wherein at least one of:
(i) one of the supports (1-9) has several one of the information carriers (11);
(ii) two adjacent or opposing sides of one of the supports (1-9) each has one of the information carriers (11); or
(iii) the information carriers (11) of the supports (1-9) having several information carriers (11) additionally comprise information regarding a side to which the respective information carrier (11) is fastened.

18. A first one of the supports (1-9) for implementing the method of claim 1, the first support comprising a base section (10) for placement on one of the support elements (21).

19. The first support of claim 18, wherein the information of the information carrier (11) of the first support further comprises one or more of a date, a type of the first support, a unique identification value, an orientation, a checksum, a batch number or a height value.

20. The first support of claim 18, wherein the information carrier (11) of the first support comprises a barcode, and the reading device is a barcode scanner.

21. The first support of claim 18, wherein at least one of:
(i) the first support has several one of the information carriers (11);
(ii) two adjacent or opposing sides of the first support each has one of the information carriers (11); or
(iii) the information carriers (11) of the first support having several information carriers (11) additionally comprise information regarding a side of the first support to which the respective information carrier (11) is fastened.
